(19) United States
(12) Patent Application Publication
(10) Pub. No.: US 2016/0314929 A1
(43) Pub. Date: Oct. 27, 2016

(54) Beam Guidance System, Particle Beam Therapy System and Method

(71) Applicants: Jürgen Drees, Wuppertal (DE); Helmut Piel, Wuppertal (DE)

(72) Inventors: Jürgen Drees, Wuppertal (DE); Helmut Piel, Wuppertal (DE)

(73) Assignee: Cryoelectra GmbH, Wuppertal (DE)

(21) Appl. No.: 15/133,347

(22) Filed: Apr. 20, 2016

(30) Foreign Application Priority Data
Apr. 23, 2015 (DE) 10 2015 106 246.1

(51) Int. Cl. H01J 37/147; A61N 5/10; H01J 37/14

(52) U.S. Cl. CPC H01J 37/147; H01J 37/14; A61N 5/1077; H01J 2237/152; H01J 2237/14; A61N 2005/1087

(57) ABSTRACT

The invention relates, inter alia, to a beam guidance system for guiding a beam of charged particles with a magnetic beam deflection unit, wherein the magnetic beam deflection unit has an entry side for entry of the beam of charged particles into the magnetic beam deflection unit in a direction of entry, wherein the magnetic beam deflection unit has an exit side for exit of the beam of charged particles from the magnetic beam deflection unit in a direction of exit. A further aspect of the invention relates to an advantageous particle beam therapy system. The problem of providing improving beam properties and at the same time reducing the amount of space required is solved in that the entry side of the magnetic beam deflection unit is, at least in sections, aligned substantially parallel to the exit side of the magnetic beam deflection unit.

### CROSS-REFERENCE TO RELATED PATENT APPLICATIONS

This patent application claims priority to German Application No. 10 2015 106 246.1, filed Apr. 23, 2015, the entire teachings and disclosure of which are incorporated herein by reference thereto.

### FIELD OF THE INVENTION

According to one aspect, the invention relates to a beam guidance system for guiding a beam of charged particles with a magnetic beam deflection unit, wherein the magnetic beam deflection unit has an entry side for entry of the beam of charged particles into the magnetic beam deflection unit in a direction of entry, and wherein the magnetic beam deflection unit has an exit side for exit of the beam of charged particles from the magnetic beam deflection unit in a direction of exit. According to a further aspect, the invention relates to an advantageous particle beam therapy system. According to a further aspect, the invention also relates to an advantageous method.

### BACKGROUND OF THE INVENTION

In the prior art, beam guidance systems of the aforementioned type are for example used in particle beam therapy systems for radiotherapy with charged particles, for example ions in the form of protons. In comparison with the previously usual forms of radiotherapy with photons, this offers significant advantages for patients with certain forms of cancer. In particular, radiotherapy with ions, in particular protons, is advantageous, since these only attain their maximum ionisation strength, and accordingly their greatest destructive power, for example for tumour cells, at the end of their path into the tissue which is being irradiated, at the so-called Bragg peak. In this way, adverse effects on healthy tissue which lies in front of the tissue which is to be treated along the path of the beam and which is passed through by the beam can be reduced. Moreover, adverse effects on healthy tissue which lies beyond the beam can be almost completely avoided.

However, the successful treatment of tumours with a particle beam, for example by means of the raster scan therapy method, requires a particle beam with precise beam properties, in particular the beam position or the beam momentum, so that a precise treatment of the tissue can take place at the treatment location (at the so-called isocentre) and inaccuracies in treatment avoided, so that as far as possible no undesired irradiation of healthy tissue (for example adjacent organs) takes place.

However, the particle beam therapy systems known from the prior art, that is to say the overall systems and in particular the beam guidance systems, take up a comparatively large amount of space. Moreover, the beam properties and the achievable transmission of the particle beam up to the treatment location offer scope for improvement.

For example, a particle beam therapy system is known from WO 2009/106603 A1 wherein a particle beam is generated and fed via a beam guidance system to one of several treatment rooms. In order to reduce operating costs it is suggested that different beam guidance systems be provided which are adjusted to particular beam properties. Although this can save space through the use of a single beam generating unit for several treatment locations, this is only possible if several treatment locations are present and a lot of space is available in any case. Moreover, the beam guidance system and the beam switches provided therein still take up a comparatively large amount of space.

A particle beam therapy system is also known from EP 2 268 359 B1 wherein in order to guarantee a precise beam guidance it is suggested that the beam properties in the halo region of the particle beam by means of a beam position monitor during treatment. Although this makes possible an improved monitoring of the beam properties, the beam properties remain limited by the technical limitations of the equipment, so that the need remains to improve the beam properties. Moreover, this system too takes up a comparatively large amount of space.

### SUMMARY OF THE INVENTION

Starting out from this prior art, it is the object of the present invention to provide a generic beam guidance system which can provide comparable or even improved beam properties while requiring less space. The present invention is also based on the object of suggesting an advantageous particle beam therapy system and an advantageous method.

According to one aspect of the present invention, this problem is solved in a generic beam guidance system with a magnetic beam deflection unit in that the entry side of the magnetic beam deflection unit is, at least in sections, aligned substantially parallel to the exit side of the magnetic beam deflection unit.

Previously, magnetic beam deflection units have been used in the prior art the entry side and exit side of which are aligned at an angle to each other, since the entry side and/or the exit side are as a rule aligned at right angles to the beam. If, for example, a magnetic beam deflection unit is provided in order to deflect the beam of charged particles by 45°, the entry side and the exit side of the magnetic beam deflection unit also enclose an angle of 45°.

The invention departs from this design and suggests a beam deflection unit the entry side of which is, at least in sections, aligned substantially parallel to the exit side. It has been found that if a beam of charged particles is to be deflected from a direction of entry into a direction of exit which is different from the direction of entry, improved beam properties and at the same time more compact beam guidance systems can be provided through the design of the beam deflection unit according to the invention which could not be achieved previously in the prior art. This is attributable, inter alia, to the fact that such magnetic beam deflection units can achieve not only a deflection of the beam of charged particles, but also a focusing of the beam of charged particles similarly to a beam forming unit, because the magneto-optical properties of a magnetic beam deflection unit (for example of a dipole magnet) are largely determined through the angle of the incident beam of charged particles in relation to the entry and exit edge. As a result, fewer beam forming units are needed in the beam guidance system as a whole. Moreover, an increased transmission of the beam of charged particles through the beam guidance system can be achieved. This means that, overall, a more compact particle beam therapy system with improved beam properties can be provided. In addition, it is simpler to manufacture beam deflection units in which the entry side is at least in sections aligned substantially parallel to the exit side, which further reduces manufacturing costs.

For example, the entry side and/or the exit side of the magnetic beam deflection unit is, at least in sections, planar in form. In this case, in particular the planar sections of the entry side and the exit side are, at least in sections, aligned substantially parallel to each other. The entry side can in particular be an end face, for example a front side of the beam deflection unit, which for example has an entry aperture for entry of the beam of charged particles. The exit side can in particular be a further end face, for example an end face opposite the entry side, for example a rear side of the beam deflection unit, which for example has an exit aperture for the exit of the beam of charged particles.

Preferably, the entire entry side and the entire exit side are substantially parallel to each other. Substantially parallel means that the entry side and the exit side enclose for example an angle of less than 5°, preferably an angle of less than 3°, particularly preferably less than 1°.

In order to deflect the beam of charged particles the magnetic beam deflection unit is for example provided in the beam guidance system such that the beam is deflected from the direction of entry into the desired direction of exit.

The magnetic beam unit is preferably designed to effect a deflection of the beam of charged particles which remains constant over time. This means that the beam of charged particles is for example continuously deflected by 45°.

More than one beam deflection unit can also be provided in the beam guidance system. For example, two, three, four or more beam deflection units are provided. If more than one beam deflection unit is provided in the beam guidance system, for example all or only some (for example one, two, three or four) of the beam deflection units can be designed according to the invention. Accordingly, the formulation a/the beam deflection unit is to be understood to mean at least one/the at least one beam deflection unit.

The beam of particles is preferably an ion beam, in particular a proton beam. The beam guidance system is for example designed to guide the beam of charged particles precisely with energies of 60 to 210 MeV. 1 MeV corresponds roughly to 1.6×10⁻¹³ Joules. The energy of the particles is in particular to be understood to refer to the average or maximum kinetic energy of the particles.

The beam deflection unit can for example be controlled such that a desired deflection is for example possible for different energies of the charged particles of the beam of charged particles.

According to a preferred embodiment of the beam guidance system according to the invention, the magnetic beam deflection unit is provided in the beam guidance system for deflection of the beam of charged particles such that the entry side lies at an oblique angle to the direction of entry of the beam of charged particles and/or the exit side lies at an oblique angle to the direction of exit of the beam of charged particles.

In the beam guidance system, the beam of charged particles can in particular enter the beam deflection unit in a defined direction of entry and exit from the beam deflection unit in a defined direction of exit. Because the entry side lies at an oblique angle to the direction of entry of the beam of charged particles and the exit side lies at an oblique angle to the direction of exit of the beam of charged particles, the beam deflection unit can in particular be arranged symmetrically in the beam of charged particles, which further improves the beam properties. An oblique arrangement is in particular understood to mean that a side is in particular not at right angles to and/or not parallel to the direction of entry or direction of exit. For example, the entry side and/or the exit side are aligned substantially parallel to the perpendicular bisector of the angle which is formed between the direction of entry and the direction of exit of the beam of charged particles. For example, the angle between the entry side and the direction of entry and between the exit side and the direction of exit are equal.

According to a preferred embodiment of the beam guidance system according to the invention, the magnetic beam deflection unit is provided in the beam guidance system for deflection of the beam of charged particles such that the direction of entry and the direction of exit are oriented at an angle of 30° to 60°, preferably of 40° to 50°, in particular substantially 45°, relative to each other.

The beam of charged particles is thus for example deflected by 30° to 60°, preferably by 40° to 50°, in particular by substantially 45°. It has been found that a deflection of the beam of charged particles by these angles can be achieved with good beam properties and at the same time a compact beam guidance system provided.

According to a preferred embodiment of the beam guidance system according to the invention, the magnetic beam deflection unit is a dipole magnet.

Through the design of the magnetic beam deflection unit as a dipole magnet, a substantially homogeneous magnetic field can, in a simple manner, be provided for the deflection of the beam of charged particles. Moreover, dipole magnets can be manufactured comparatively simply. In this case manufacture is in particular simplified in that the entry side is, at least in sections, aligned substantially parallel to the exit side. The dipole magnet is for example an electromagnet. For example, the dipole magnet has an iron core, for example an iron yoke. The iron core can for example consist of iron plates. For example, the iron core is manufactured from stacked iron plates. This makes possible a simple manufacture of the dipole magnet. The dipole magnet can for example, viewed in the direction of the beam of charged particles, have a distance between the substantially parallel entry side and exit side of 0.5 to 2 m, preferably around 1 m.

It has been found that the use of dipole magnets in the beam guidance system wherein the entry side is, at least in sections, aligned substantially parallel to the exit side makes it possible to achieve good beam properties. This is attributable to the fact that, through the design according to the invention, the dipole magnets can, in a similar way to quadrupole magnets, assist the focusing of the beam of charged particles.

According to a preferred embodiment of the beam guidance system according to the invention, the beam guidance system has several, in particular four magnetic beam deflection units.

If a plurality of magnetic beam deflection units are provided which are designed according to the invention, a more complex path of the beam of charged particles can be achieved in a compact beam guidance system. As already explained, at least some, preferably all of the plurality of magnetic beam deflection units are designed according to the invention.

Preferably, the plurality of beam deflection units are provided in the beam guidance system such that the beam of charged particles (at a particular point in time) substantially runs in a plane.

For example, the beam guidance system is configured such that the beam of charged particles is deflected through the first beam deflection unit from the original axis of the beam of charged particles and runs obliquely to the original axis. The original axis of the beam of charged particles is for example the axis along which the beam of charged particles moves before the first beam deflection unit. For example, the beam guidance system is configured such that the beam of charged particles is deflected through the further magnetic beam deflection units (for example a second, third and fourth magnetic beam deflection unit) back onto the original axis of the beam of charged particles. For example, the beam guidance system is configured such that after the second beam deflection unit the beam of charged particles runs parallel to the original axis of the beam of charged particles. For example, the beam guidance system is configured such that after the last (for example the fourth) beam deflection unit the beam of charged particles runs transversely, in particular substantially at right angles, to the original axis of the beam of charged particles and preferably intersects the original axis.

Preferably, at least some of the several magnetic beam deflection units are identical in design. As a result, the manufacturing costs for the magnetic beam deflection units can be reduced. For example, all but one of the magnetic beam deflection units (for example the last magnetic beam deflection unit before the treatment location) are identical in design. For example, the fourth beam deflection unit is of different design. A different design of in particular the last magnetic beam deflection unit makes possible an adjustment to the conditions in the beam path. For example, the fourth beam deflection unit has an enlarged entry aperture and/or exit aperture in comparison with the other magnetic beam deflection units in order to provide an irradiation of a sufficient volume at the treatment location.

For example, the second and third magnetic beam deflection units, viewed in the direction of the beam of charged particles, can be spaced 0.5 m to 2 m, preferably 1 m to 1.5 m apart. For example, the last magnetic beam deflection unit, viewed in the direction of the beam of charged particles, is at most 1.5 m, preferably at most 1 m distant from the treatment location. The distance between two magnetic beam deflection units is in particular understood to refer to the distance from the exit point of the beam of charged particles from one magnetic beam deflection unit to the entry point of the beam of charged particles into the other magnetic beam deflection unit.

The beam of charged particles can be deflected, by means of the beam guidance system for example, over a distance of less than 10 m, in particular less than 8 m, from the direction running along the original axis of the beam of charged particles into a direction at right angles thereto and intersecting the original axis.

According to a preferred embodiment of the beam guidance system according to the invention, the beam guidance system has at least one magnetic beam forming unit, in particular several magnetic beam forming units, preferably in the form of one or several quadrupole magnets.

Through the provision of magnetic beam forming units, in particular in the form of quadrupole magnets, the beam property can be further improved and the beam of charged particles efficiently focused. It has been found that comparatively few beam forming units are necessary in the beam guidance system in order to achieve good beam properties, which makes possible a compact beam guidance system. Generally however, other beam forming units can also be provided. For example, beam forming units in the form of sextupole magnets can be provided.

Due to their structure, quadrupole magnets can only focus a beam of charged particles in a direction transverse to the beam of charged particles. In this respect it is advantageous to provide at least two quadrupole magnets in order to achieve a beam formation in both directions (i.e. in the plane) transverse to the beam of charged particles.

For example, at least five and/or at most ten, preferably seven beam forming units are provided in the beam guidance system. For example, at least four and/or at most six, preferably five beam forming units are provided between the (viewed in the direction of the beam of charged particles) first and the second magnetic beam deflection unit. For example, two beam forming units are provided between the second and the third magnetic beam deflection unit. However, further beam forming units can also be provided. For example, some, preferably all of the beam forming are of equal dimensions. This reduces the manufacturing costs of the beam guidance system.

According to a further preferred embodiment of the beam guidance system according to the invention, the beam guidance system has a collimator unit before the first magnetic beam deflection unit, viewed in the direction of the beam of charged particles.

As a result, the beam properties are improved while requiring little additional space. Through the collimator unit, the phase space of the beam of charged particles transported after the collimator unit can be limited. The phase space of the beam of charged particles is in particular determined through the location and momentum of the particles. As a rule, the higher the phase space density, the narrower the beam profile and the lower the divergence of the particle beam. A beam with high phase space density requires less beam guidance in comparison with a particle beam with lower phase space density.

For example, the collimator unit is designed as a screen, for example as a block of material with one or more apertures. For example, the collimator unit has an angular, for example rectangular aperture. Preferably, the aperture can be altered in both directions transverse to the beam of charged particles. This makes possible a flexible adjustment of the beam properties.

According to a further preferred embodiment of the beam guidance system according to the invention, the beam guidance system has at least two magnetic beam deflection units, and has a collimator unit between two of the at least two magnetic beam deflection units.

It has been found that a large momentum dispersion can prevail, in particular between two beam deflection units. Through the provision of a collimator unit, the beam properties can be further improved while requiring little additional space. It has been found that a comparatively large momentum dispersion prevails in particular between the second and third beam deflection unit, so that the beam properties can be especially improved through the provision of a collimator unit. The collimator unit can therefore be used in particular to achieve a momentum selection for the beam of charged particles at the treatment location. As already described, the collimator unit can for example, be designed as a screen, for example as a block of material with one or more apertures. For example, the collimator unit has an angular, for example rectangular aperture. Preferably, the aperture can be altered in both directions transverse to the beam of charged particles. This makes possible a flexible adjustment of the beam properties. In particular, the shape of the beam spot at the treatment location can be adjusted. Moreover, the losses after the collimator up to the treatment location can be limited to less than 1%, even though further, for example a further two beam deflection units are arranged in the beam guidance system, viewed in the direction of the beam.

The beam guidance system can also include further units which are not mentioned here. For example, a scanning magnet can be provided in the beam guidance system. For example, the scanning magnet is provided between two beam deflection units. Preferably, the scanning magnet is provided between the last and the last but one (for example, between the third and the fourth) beam deflection unit, since in this position the scanning magnet advantageously makes possible a large scanning area at the treatment location.

As a further example of a further unit, the beam guidance system can include one or more beam monitors. Beam monitors can be used to measure the beam properties, for example the beam position and the particle momentum of the beam of charged particles, in particular at different points.

According to a further preferred embodiment of the beam guidance system according to the invention, the beam guidance system is, at least in sections, designed to be movable, in particular rotatable.

For example, the beam guidance system can have an immoveable section and a moveable section. For example, in order to realise the moveable section the beam guidance system has a moveable supporting frame, a so-called gantry. The supporting frame can advantageously be rotatable by up to 360°, in particular around a horizontal axis, in order to allow the treatment location to be irradiated from as many angles as possible. The axis of rotation of the supporting frame coincides in particular with the original axis of the beam of charged particles (i.e. in particular with the axis of the beam of charged particles prior to deflection through the first beam deflection unit).

The moveable section (in particular the gantry) can for example include all or some of the aforementioned elements of the beam guidance system, in particular one or more (for example four) beam deflection units, one or more (for example seven) beam forming units, one or more (for example two) collimator units, one or more scanning magnets and/or one or more beam monitors.

Generally, it is also conceivable to design the moveable section of the beam guidance system free of collimator units. This allows the beam guidance system to be of more compact design.

For example, the moveable section of the beam guidance system can be designed such that the beam of charged particles is fed to the treatment location in such a way that the beam of charged particles is a maximum of 3 m distant from the original axis of the beam of charged particles.

According to a preferred embodiment of the beam guidance system according to the invention, the beam guidance system has an energy correction unit for adjustment of the energy of the charged particles of the beam.

The energy correction unit makes possible a flexible adjustment of the energy of the charged particles of the beam of charged particles. For example, it is possible to use a beam generating unit (for example a cyclotron) which emits charged particles with substantially constant energy without needing to dispense with the possibility of being able to adjust the energy of the charged particles of the beam of charged particles. For example, the energy correction unit is configured to reduce the energy of the charged particles of the beam of charged particles. For example, the energy correction unit is a degrader. For example, only one beam generating unit, which emits particles with sufficient maximum energy, needs to be provided. By means of the energy correction unit, the energy of the charged particles of the beam of charged particles can then be reduced as required.

For example, the energy of the charged particles of the beam of charged particles is reduced in that the (average) kinetic energy of the charged particles is reduced. The energy correction unit can for example, be used to reduce the charged particles to a selectable average kinetic energy.

It has been found that the beam guidance system can be built so compact that, for example, the treatment location can be positioned less than 10 m, in particular less than 9 m from the end of the energy correction unit.

For example, a beam generating unit can be used which emits charged particles with a kinetic energy of more than 200 MeV, for example between 200 and 300 MeV, in particular between 210 and 250 MeV. The energy correction unit can for example be such that the kinetic energy of the charged particles can for example be reduced to below 200 MeV or to below 100 MeV.

Preferably, the beam of charged particles passes through the energy correction unit at least partially in a vacuum (for example, at least a fine vacuum).

The energy correction unit is preferably provided in the immoveable section (in particular not in the moveable supporting frame) of the beam guidance system.

According to a preferred embodiment of the beam guidance system according to the invention, the energy correction unit has at least one block-formed energy correction element which can be displaced transversely to the beam of charged particles and at least one wedge-formed energy correction element which can be displaced transversely to the beam of charged particles.

A block-formed energy correction element comprises, for example, an entry side and an exit side for the beam of charged particles which are aligned substantially parallel. A block-formed energy correction element can for example be a cube.

A wedge-formed energy correction element comprises, for example, an entry side and an exit side for the beam of charged particles which are aligned obliquely, i.e. not parallel, to each other.

A compact and precise reduction in the energy of the charged particles of the beam of charged particles can be achieved in particular through the advantageous combination of at least one block-formed and at least one wedge-formed energy correction element. In particular due to its compactness (i.e. a short distance to be travelled by the beam of charged particles), an excessive widening of the phase space of the beam of charged particles can be avoided through such an energy correction unit. The block-formed energy correction element serves, for example, to achieve a first coarse adjustment of the energy of the charged particles of the beam of charged particles. For example, the energy of the charged particles of the beam of charged particles can be adjusted to discrete values. The wedge-formed energy correction element serves, for example, to achieve a finer adjustment of the energy of the charged particles of the beam of charged particles in comparison with the first adjustment. For example, the energy of the charged particles of the beam of charged particles can be adjusted continually (for example within a particular range). For example, the extension of the wedge-formed energy correction element into the region of the beam of charged particles, viewed in the direction of the beam of charged particles, is altered through the displacement of the wedge-formed energy correction elements transversely to the beam of charged particles. Through the arrangement of the block-formed energy correction elements before the wedge-formed energy correction elements, viewed in the direction of the beam of charged particles, the latter can be correspondingly shorter in design, since the energy only needs to be adjusted over a narrower range.

For example, the at least one block-formed energy correction element is displaceable in a direction (for example in the x-direction) transverse to the beam of charged particles. For example, the at least one wedge-formed energy correction element is displaceable transversely to the beam of charged particles in the same transverse direction and/or in the direction at right angles thereto (e.g. in the x-direction and/or in the y-direction).

Preferably, the at least one block-formed energy correction element and the at least one wedge-formed energy correction element are displaceable substantially at right angles to the beam of charged particles.

According to a further separate aspect of the invention, the energy correction unit according to this embodiment is also disclosed isolated from the other aspects. In particular, an energy correction unit is disclosed wherein the energy correction unit has at least one block-formed energy correction element displaceable transversely to the beam of charged particles and at least one wedge-formed energy correction element displaceable transversely to the beam of charged particles. According to a further separate aspect of the invention, a beam guidance system is also disclosed wherein the beam guidance system has an energy correction unit for adjustment of the energy of the charged particles of the beam and the energy correction unit has at least one block-formed energy correction element displaceable transversely to the beam of charged particles and at least one wedge-formed energy correction element displaceable transversely to the beam of charged particles. However, the energy correction unit and the beam guidance system according to these separate aspects can be further configured according to the previous and following advantageous embodiments.

According to a preferred embodiment of the beam guidance system according to the invention, the energy correction unit has a plurality of block-formed energy correction elements displaceable transversely to the beam of charged particles which make possible different adjustments to the energy of the beam of charged particles.

A flexible adjustment of the energy of the charged particles of the beam of charged particles over a wide energy range can be made possible through the plurality of (preferably different) block-formed energy correction elements. The block-formed energy correction elements can for example have different dimensions of extension, viewed in the direction of the beam of charged particles, and/or consist of different materials.

According to a further preferred embodiment of the beam guidance system according to the invention, the energy correction unit has several, in particular two wedge-formed energy correction elements displaceable transversely to the beam of charged particles.

For example, the beam guidance system is configured such that the wedge-formed energy correction elements can be positioned simultaneously in the beam of charged particles. For example, a wedge-formed energy correction element can be displaced from a first direction into the beam of charged particles and a further wedge-formed energy correction element can be displaced from a second direction (for example from a direction opposite to the first direction) into the beam of charged particles.

This allows the range within which a fine adjustment is possible to be expanded. Moreover, an asymmetrical reduction in the energy over the cross section of the beam of charged particles can be avoided in this way. For example, for this purpose two wedge-formed energy correction elements are arranged mirror-symmetrically or point-symmetrically in relation to each other.

According to a further preferred embodiment of the beam guidance system, the energy correction unit is at least partially manufactured of boron carbide.

In contrast to the materials used in the prior art, a more compact and reliable energy correction unit is provided through the use of boron carbide (B₄C). The high content of boron (atomic number 5) and the high density of boron carbide reduce the widening of the phase space in comparison with previously used materials. For example, in comparison with energy correction units consisting (exclusively) or graphite, an up to 30% more compact energy correction unit can be provided. Preferably, the distance from the exit window of the beam generating unit (for example an accelerator) to the end of the energy correction unit, viewed in the direction of the beam of charged particles, is less than 2 m, in particular at most 1.7 m. At the same time, through the lesser extension of the energy correction unit, a lesser extension in the direction of the beam of charged particles (and) a lesser widening of the beam of charged particles can be achieved. Ultimately, this makes it possible to achieve improved beam properties and improved transmission properties of the beam guidance system. Moreover, in comparison with an energy correction unit containing beryllium, an energy correction unit with lesser toxicity can be provided.

It is also possible to provide an energy correction unit consisting of different materials, for example boron carbide and graphite. For example, the block-formed and wedge-formed energy correction elements can consist of different materials.

For example, inter alia through the use of such an energy correction unit, the beam of charged particles with an energy of 215 MeV can be reduced to 90 MeV, whereby a transmission of almost 1% can be achieved at the treatment location. Even with a reduction to 60 MeV, a transmission of around 0.3% can still be achieved. In comparison with values from the prior art, these are significantly higher values.

According to a further preferred embodiment of the beam guidance system according to the invention, the beam guidance system has an a collimator unit after the energy correction unit, viewed in the direction of the beam of charged particles.

This allows the phase space to be restricted after the energy correction unit and allows the beam quality at the treatment location to be improved. Preferably, the collimator unit is arranged directly after the energy correction unit. For example, the collimator unit is designed as one or more, preferably two screens. A screen is for example, a material block with one or more apertures. For example, the screens each have a circular and/or, viewed contrary to the direction of the beam of charged particles, conically narrowing aperture. The beam of charged particles preferably passes through the collimator unit in a vacuum.

The collimator unit is preferably provided in the immoveable section (in particular not in the moveable supporting frame) of the beam guidance system.

According to a further preferred embodiment of the beam guidance system, the beam guidance system has a drift distance between the energy correction unit and the, viewed in the direction of the beam of charged particles, first magnetic beam deflection unit which is free of magnetic beam deflection units and/or magnetic beam forming units.

Through the drift distance, a region can advantageously be provided which can for example accommodate one or more measuring devices (in particular beam monitors) for monitoring the beam. This region can also be used for shielding (for example by means of a concrete shield) of the treatment location from the beam generating unit. For example, the length of the drift distance is at least 1 m and/or at most 2 m. The drift distance is preferably at least partially provided in a vacuum, for example in a vacuum tube of preferably a few centimetres in diameter. Preferably, the drift distance is free of any magnetic components.

The drift distance is preferably provided in the immoveable section (in particular not in the moveable supporting frame) of the beam guidance system.

According to a further separate aspect of the invention, the drift distance according to this embodiment is also disclosed isolated from the other aspects. In particular, a beam guidance system with an energy correction unit and at least one beam deflection unit are disclosed wherein the beam guidance system has a drift distance between the energy correction unit and the, viewed in the direction of the beam of charged particles, first magnetic beam deflection unit which is free of magnetic beam deflection units and/or magnetic beam forming units. However, the beam guidance system according to this aspect can be further configured according to the previous and following advantageous embodiments.

According to a further aspect of the present invention, the problem named in the introduction is solved through a particle beam therapy system with a beam generating unit for generating a beam of charged particles, in particular ions, preferably protons, and with a beam guidance system according to the different aspects, wherein a beam forming unit is preferably provided after the beam generating unit, viewed in the direction of the beam of charged particles.

As explained with reference to the beam guidance system according to the invention, using such a beam guidance system, particle beam therapy systems with improved beam properties and at the same time more compact construction designs can be provided which could not be achieved previously in the prior art.

For example, a beam generating unit with a diameter of less than 4 m is used. The distance from the beam generating unit up to the end of the energy correction unit can, advantageously, be less than 2 m. The distance from the end of the energy correction unit up to the treatment location can preferably be less than 9 m. Through the different aspects it is therefore possible for the length of the particle beam therapy system, measured up to the treatment location, to be less than 14 m.

For example, the beam generating unit is an accelerator device, for example an accelerator device which generates a beam of charged particles with constant energy, for example a cyclotron. However, it is also conceivable that the beam generating device is an accelerator device which generates a beam of charged particles with variable energy, for example a synchrotron. As already stated, a beam generating unit is preferably used which emits charged particles with a kinetic energy of more than 200 MeV, for example between 200 and 300 MeV, in particular between 210 and 250 MeV.

If at least one beam forming unit is provided after the beam generating unit, viewed in the direction of the beam of charged particles, the beam properties and/or the transmission can be improved. This is attributable to the fact that, through at least one beam forming unit, a projection of the phase space emitted by the beam generating unit onto the energy correction unit preferably arranged (immediately) after this can take place such that the largest possible part (ideally all) of the phase space contains charged particles which contribute to the transmission to the treatment location. As explained above, the beam forming units can in particular be magnetic quadrupoles (for example three).

A solenoid magnet can also be provided after the beam generating unit as a beam forming unit.

According to a further separate aspect of the invention, the particle beam therapy system in this embodiment is also disclosed isolated from the other aspects. In particular, a particle beam therapy system is disclosed with a beam generating unit for generating the beam of charged particles, in particular ions, preferably protons, and with a beam guidance system wherein at least one beam forming unit is provided after the beam generating unit, viewed in the direction of the beam of charged particles. However, the particle beam therapy system according to this separate aspect can be further configured according to the previous and following advantageous embodiments.

According to a further aspect of the invention, the problem named in the introduction is solved through a method comprising the steps: generation of a beam of charged particles, in particular ions, preferably protons, and guidance of the beam of charged particles by means of a beam guidance system according to the different described aspects.

With regard to the advantages and further embodiments of the methods, reference is made to the previous aspects and their embodiments.

In particular, the corresponding method step is disclosed through the previous and following description of means for carrying out a method step. Also, corresponding means or equipment for carrying out the method steps will be disclosed through the disclosure of method steps.

The examples and exemplary embodiments of all aspects of the present invention described above should also be understood to be disclosed in all combinations.

### BRIEF DESCRIPTION OF THE DRAWINGS

Further advantageous exemplary embodiments of the different aspects are explained in the following detailed description of a number of exemplary embodiments of the aspects, in particular in combination with the figures. However, the figures enclosed with the application are only intended to serve the purpose of illustration, not to define the scope of protection of the invention. The enclosed drawings are not necessarily true to scale and are simply intended to reflect in exemplary form the general concept of the present aspects. In particular, features which are contained in the figures should in no way be considered as a necessary element of the present invention.

In the drawings:

FIG. 1 shows a schematic sectional view of an exemplary embodiment of a particle beam therapy system with an exemplary embodiment of a beam guidance system according to the present invention;

FIG. 2 shows a schematic sectional view of a further exemplary embodiment of the moveable section of a beam guidance system according to the present invention; and

FIG. 3 shows a schematic sectional view of an exemplary embodiment of an energy correction unit.

### DETAILED DESCRIPTION OF THE INVENTION

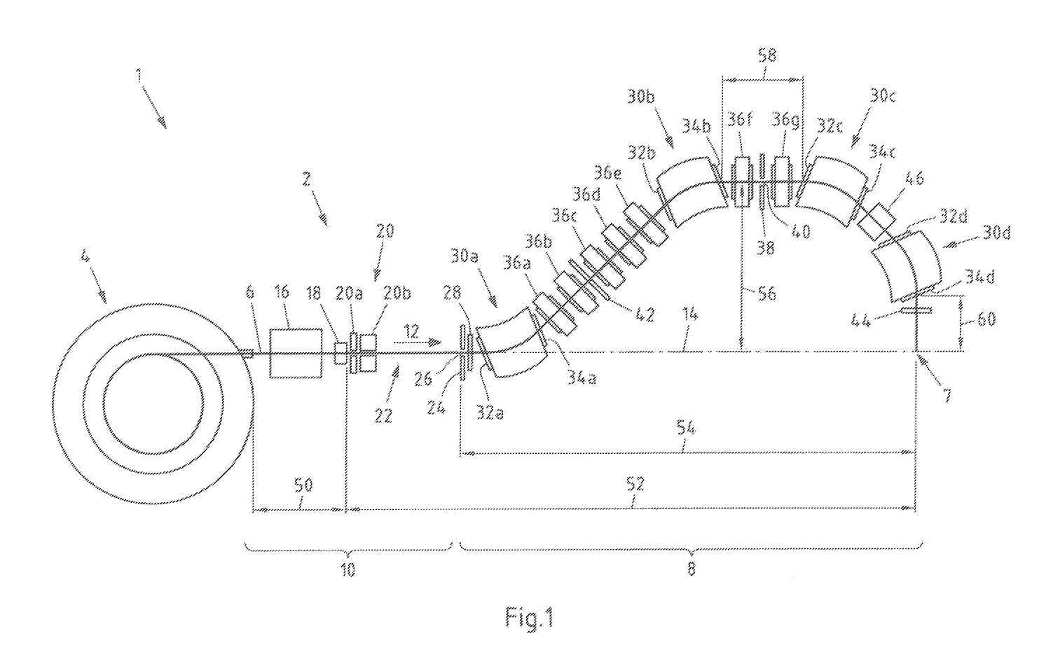

FIG. 1 shows a schematic sectional view of an exemplary embodiment of a particle beam therapy system 1 with an exemplary embodiment of a beam guidance system 2. The particle beam therapy system comprises a beam generating unit 4 for generating a beam of charged particles 6, which can in particular be protons. The beam of charged particles 6 is passed through the beam guidance system 2 to a treatment location 7. The beam guidance system 2 has a moveable, in this case rotatable by 360°, section 8 and an immoveable section 10. The rotatable section 8 can for example be realised through a supporting frame in the form a gantry (not shown).

The beam generating unit 4 is in this case a cyclotron, that is to say an accelerator device which generates the beam of charged particles 6 with constant energy. The beam generating unit 4 emits charged particles with a constant kinetic energy, for example, 210 MeV, 215 MeV or 250 MeV. It has been found that around 95% of the patients who are to be treated can be treated by means of charged particles with a kinetic energy of around 207 MeV, and still around 90% by means of charged particles with a kinetic energy of around 198 MeV.

The beam of charged particles initially runs in the direction of the arrow 12 along an original axis 14 of the beam of charged particles 6.

A beam forming unit or beam forming units 16 is, for example, provided after the beam generating unit 4, viewed in the direction 12 of the beam of charged particles 6. The beam forming unit 16 can for example be realised through one or more solenoid magnets or through several (for example three) quadrupole magnets. The focusing through the beam forming unit 16 serves to make possible an optimal projection of the phase space emitted by the beam generating unit onto the energy correction unit arranged after it such that the largest possible part (ideally all) of the phase space contains charged particles which contribute to the transmission to the treatment location. In this way the beam quality and transmission of the beam of charged particles 6 to the treatment location 7 can be increased.

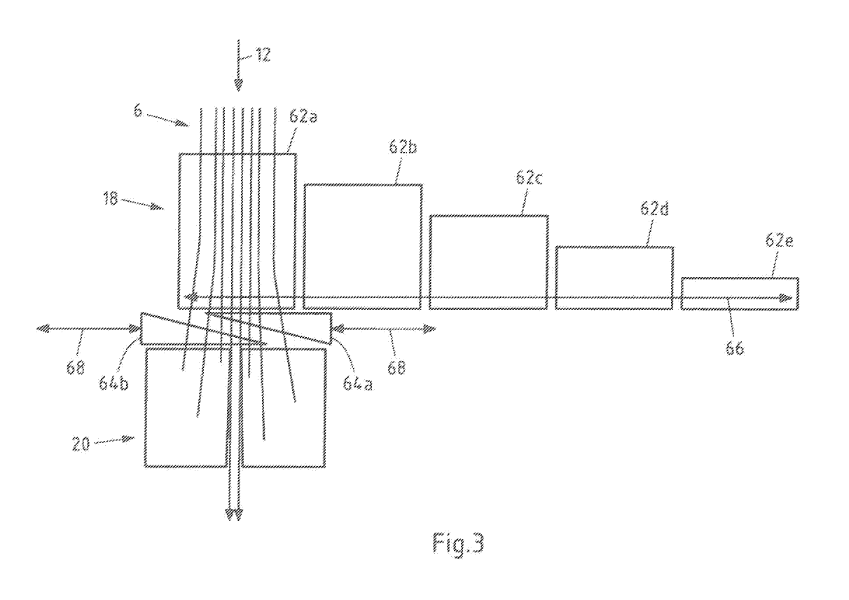

The beam of charged particles 6 is then passed through an energy correction unit 18. An adjustment of the energy of the charged particles of the beam of charged particles 6 can take place through the energy correction unit 18. The energy correction unit is described in more detail in connection with FIG. 3.

The beam guidance system also includes a collimator unit 20 arranged after the energy correction unit 18, viewed in the direction 12 of the beam of charged particles 6, which the beam of charged particles 6 then passes through. The collimator unit comprises two screens 20a, 20b which are formed respectively as a material block with a circular aperture narrowing conically contrary to the direction 12 of the beam of charged particles 6.

The beam guidance system 2 has a drift distance 22 after the energy correction unit 18 and the collimator unit 20, viewed in the direction of 12 of the beam of charged particles 6. The drift distance 22 is free of magnetic units such as magnetic beam deflection units or magnetic beam forming units. A shield, for example a concrete shield (not shown) can be provided in the region of the drift distance 22. Moreover, measuring devices such as beam monitors (not shown) can be provided in the region of the drift distance 22.

The beam of charged particles 6 passes through the section of the energy correction unit 18, the collimator unit 20 and the drift distance 22 of the beam guidance system 2 in a vacuum, which improves the beam properties and the transmission in this section.

The previously described elements of the beam guidance system 2 are arranged in the immoveable section 10 of the beam guidance system 2. The rotatable section 8 is provided in order to allow the treatment location 7 to be irradiated from as many angles as possible. The axis of rotation of the supporting frame (not shown) coincides with the original axis 14 of the beam of charged particles 6.

In the rotatable section 8, the beam guidance system first includes a collimator unit 24. The collimator unit 24 can be used to define the phase space of the beam of charged particles. The collimator unit 24 is designed here as a screen, in this case as a material block with a rectangular aperture 26. The geometry of the aperture 26 can be altered in both directions transverse to the beam of charged particles 6, so that a flexible adjustment of the beam properties can take place.

An optional beam monitor 28 is then provided in order to monitor the beam properties. However, the beam monitor can also be provided at other points in the beam guidance system 2.

In order to guide the beam of charged particles 6 the beam guidance system 2 then includes several, in this case four magnetic beam deflection units 30a, 30b, 30c, 30d designed as magnetic dipoles. The magnetic beam deflection units 30a, 30b, 30c, 30d each have an entry side 32a, 32b, 32c, 32d for entry of the beam of charged particles 6 in a direction of entry into the relevant magnetic beam deflection unit. The magnetic beam deflection units 30a, 30b, 30c, 30d also have an exit side 34a, 34b, 34c, 34d for the exit of the beam of charged particles 6 in a direction of exit from the magnetic beam deflection unit 30a, 30b, 30c, 30d. The entry sides 32a, 32b, 32c, 32d are each aligned parallel to the associated exit side 34a, 34b, 34c, 34d. For deflection of the beam of charged particles 6, the magnetic beam deflection units 30a, 30b, 30c, 30d are each arranged in the beam guidance system 2 such that the respective entry side 32a, 32b, 32c, 32d is aligned obliquely to the direction of entry of the beam of charged particles 6 and the respective exit side 34a, 34b, 34c, 34d is aligned obliquely to the respective direction of exit of the beam of charged particles 6. The magnetic beam deflection units 30a, 30b, 30c, 30d are in this case arranged in the beam guidance system 2 for deflection of the beam of charged particles 6 such that in each beam deflection unit 30a, 30b, 30c, 30d the direction of entry and the direction of exit are aligned at an angle of 45° relative to each other. The beam deflection units 30a, 30b, 30c, 30d are thereby positioned symmetrically in the beam of charged particles, that is to say the angle between direction of entry and entry side 32a, 32b, 32c, 32d and the angle between direction of exit and exit side 34a, 34b, 34c, 34d are in each case identical in the individual beam deflection units 30a, 30b, 30c, 30d.

The beam of charged particles 6 is deflected from the original axis 14 of the beam of charged particles through the first beam deflection unit 30a. The beam of charged particles 6 is deflected back into the direction of the original axis 14 through the second beam deflection unit 30b and then runs parallel to the original axis 14 of the beam of charged particles 6. The beam of charged particles 6 is then deflected into the direction of the original axis 14 through the third and the fourth beam deflection unit 30c, 30d, so that after the last beam deflection unit 30d the beam of charged particles 6 runs at right angles to the original axis 14 of the beam of charged particles 6 and intersects the original axis 14.

The described design of the beam deflection units 30a, 30b, 30c, 30d allows improved beam properties to be achieved with an at the same time more compact beam guidance system 2. This is, inter alia, attributable to the fact that the magnetic beam deflection units 30a, 30b, 30c, 30d can achieve not only a deflection of the beam of charged particles but also a focusing of the beam of charged particles similarly to a quadrupole magnet.

The provided magnetic beam deflection units 30a, 30b, 30c, 30d thereby display a defocusing in a transverse direction (here in the drawing plane) and a focusing in a direction at right angles to this. In this respect, the magnetic beam deflection units 30a, 30b, 30c, 30d have similar properties to quadrupole magnets, which also focus in a transverse direction and defocus in the direction at right angles to this. All four magnetic beam deflection units 30a, 30b, 30c, 30d also only focus in one direction (at right angles to the drawing plane). Of the seven quadrupole magnets provided, five therefore focus in the transverse direction in the drawing plane and only two in the direction at right angles to this. Overall, an adequate focusing in both transverse coordinate directions is achieved in this way. Effectively, through the focusing of the dipoles in the y-direction, only 2 further quadrupoles with focusing in the same direction are necessary.

Moreover, due to the parallel entry and exit sides, the manufacturing method for the beam deflection units 30a, 30b, 30c, 30d can be simplified, since the iron core of the beam deflection units 30a, 30b, 30c, 30d can be manufactured through plates which are stacked parallel on top of each other.

The beam guidance system 2 also includes several, in this case seven, magnetic beam forming units 36a, 36b, 36c, 36d, 36e, 36f, 36g in the form of quadrupole magnets. The beam properties of the beam of charged particles 6 can be further improved through the beam forming units 36a, 36b, 36c, 36d, 36e, 36f, 36g. In particular, inter alia due to the advantageous beam deflection units 30a, 30b, 30c, 30d, only a comparatively small number of beam forming units 36a, 36b, 36c, 36d, 36e, 36f, 36g are necessary in order to achieve good beam properties, which makes possible a compact beam guidance system 2.

The five beam forming units 36a, 36b, 36c, 36d, 36e are provided between the first magnetic beam deflection unit 30a and the second magnetic beam deflection unit 30b (viewed in the direction 12 of the beam of charged particles 6). A further two beam forming units 36f, 36g are provided between the second magnetic beam deflection unit 30b and the third magnetic beam deflection unit 30c. The beam forming units 36a, 36b, 36c, 36d, 36e, 36f, 36g are in this case all of equal dimensions.

The beam guidance system 2 includes a further collimator unit 38 between the two magnetic beam deflection units 30b, 30c in the form of a screen with a rectangular aperture 40. The aperture can be altered in both directions transverse to the beam of charged particles 6, allowing the shape of the beam spot at the treatment location 7 to be adjusted. It has been found that a comparatively large momentum dispersion prevails between the beam deflection units 30b, 30c. This can be counteracted through the provision of the collimator unit 38. This is because the collimator unit 38 allows a momentum selection for the beam of charged particles 6 to be achieved at the treatment location 7.

The beam guidance system 2 also includes the beam monitors 42 and 44. The beam monitor 42 is arranged between the beam forming units 36b and 36c. The beam monitor 44 is arranged after the fourth beam deflection unit 30d and before the treatment centre 7.

The beam guidance system also includes a scanning magnet 46 between the beam deflection unit 30c and the beam deflection unit 30d. The scanning magnet can be used advantageously in this position since this allows a larger scanning area to be covered at the treatment location. With a system such as, for example, that already presented in 2005 by V. Anferov, the beam can for example be displaced such that an area of 210 mm by 175 mm can be covered at the treatment location, with an angle of deflection of only ±44 mrad in both coordinate directions. A further enlargement of the scanning area is possible. Since the beam deflection unit 30d has to absorb the charged particles 6 deflected by the scanning magnet 46, the beam deflection unit 30d can have an enlarged entry aperture and/or exit aperture in comparison with the other beam deflection units 30a, 30b, 30c. The beam deflection units 30a, 30b, 30c can be of identical construction design.

The fact that the scanning magnet 46 is arranged between the last and last but one (i.e. the third and the fourth) beam deflection unit 30c, 30d makes it possible to cover a larger scanning area at the treatment location 7.

It has been found that the beam guidance system 2 can be made particularly compact. The distance 50 from the beam generating unit 4 to the end of the energy correction unit 18 is here less than 2 m. The distance 52 from the end of the energy correction unit 18 to the treatment location 7 is here less than 9 m. In this case the beam of charged particles 6 can be guided with the beam guidance system 2 over a distance 54 of less than 8 m from the direction 12 along the original axis 14 of the beam of charged particles 6 to the treatment location 7. The maximum distance 56 of the beam of charged particles 6 is thereby less than 3 m from the original axis 14 of the beam of charged particles 6. The distance 58 between the second and third magnetic beam deflection units 30b, 30c is thereby less than 1.5 m. The distance 60 from the last magnetic beam deflection unit 30d to the treatment location is thereby less than 1 m, for example 0.991 m.

It should be noted that the geometrical dimensions relate to a beam of charged particles with a kinetic energy of around 210 MeV. If higher energies are used, the geometrical dimensions are preferably multiplied by a factor. This geometrical scaling factor is for example just the ratio of the momentum of protons of higher energy (for example 245 MeV) to 210 MeV protons.

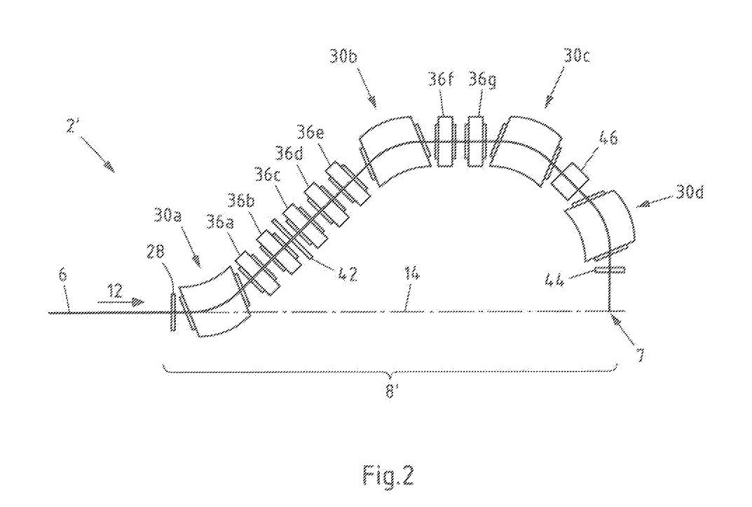

FIG. 2 shows a schematic sectional view of a further exemplary embodiment of a beam guidance system 2′. The rotatable section 8′ of the beam guidance system 2′ shown in FIG. 2 is similar to the rotatable section 8 of the beam guidance system 2 from FIG. 1. Accordingly, the same reference numbers are used for identical elements. Reference is also made to the remarks relating to the beam guidance system 2 shown in FIG. 1. In particular, the rotatable section 8′ can be provided in the beam guidance system 2 instead of the rotatable section 8. Only the differences in comparison with the beam guidance system 2 from FIG. 1 will be discussed in the following.

The main difference between the beam guidance system 2 and the beam guidance system 2′ is that the beam guidance system 2′ does not have any collimator units 24, 38 in the rotatable section 8′. In particular, this makes it possible to reduce the distance between the magnetic beam deflection units 30b, 30c, so that said distance can for example be less than 1.2 m, in particular less than 1.1 m. In this case the selection of the phase space already takes place before the beam of charged particles 6 enters the rotatable section 8′.

FIG. 3 shows a schematic sectional view of an exemplary embodiment of an energy correction unit 18 in the form of a degrader, such as can be used, for example, in the beam guidance system 2 or 2′. The energy correction unit 18 has a plurality of block-formed energy correction elements 62a, 62b, 62c, 62d, 62e which are displaceable transversely to the beam of charged particles 6 and two wedge-formed energy correction elements 64a, 64b which are displaceable transversely to the beam of charged particles 6.

The block-formed energy correction elements 62a, 62b, 62c, 62d, 62e are in this case displaceable along the arrow 66 at right angles to the beam of charged particles 6. This makes it possible to carry out different adjustments to the energy of the charged particles of the beam of charged particles 6, depending on which of the block-formed energy correction elements 62a, 62b, 62c, 62d, 62e is moved into the beam of charged particles. For this purpose, the block-formed energy correction elements 62a, 62b, 62c, 62d, 62e extend by different lengths, viewed in the direction 12 of the beam of charged particles 6. In this case the block-formed energy correction elements 62a, 62b, 62c, 62d, 62e serve to allow a coarse adjustment of the energy of the charged particles of the beam of charged particles 6 in that the energy of the charged particles of the beam of charged particles 6 can be adjusted to discrete values through the block-formed energy correction elements 62a, 62b, 62c, 62d, 62e.

The wedge-formed energy correction elements 64a, 64b can also be displaced along the arrows 68 at right angles to the beam of charged particles 6. The wedge-formed energy correction element 64a, 64b allow a fine adjustment of the energy of the charged particles of the beam of charged particles 6 after the beam of charged particles 6 has been passed through one of the block-formed energy correction elements 62a, 62b, 62c, 62d, 62e. The energy of the charged particles of the beam of charged particles 6 can be continuously adjusted within a limited range through the wedge-formed energy correction element 64a, 64b. The extension of the wedge-formed energy correction elements 64a, 64b into the area of the beam of charged particles, viewed in the direction of the beam of charged particles 6, can be adjusted through the displacement of the wedge-formed energy correction elements transversely to the beam of charged particles. The two wedge-formed energy correction elements 64a, 64b are in this case arranged point-symmetrically in relation to one another. The oblique surfaces of the wedge-formed energy correction elements 64a, 64b face one another. This arrangement allows an asymmetrical reduction in the energy over the cross section of the beam of charged particles 6 to be avoided.

The block-formed energy correction elements 62a, 62b, 62c, 62d, 62e of the energy correction unit 18 are manufactured of graphite and/or of boron carbide. However, it is also conceivable for block-formed energy correction elements made of different materials to be provided. The wedge-formed energy correction elements 64a, 64b of the energy correction unit 18 are manufactured of graphite and/or of boron carbide. Here too, it is conceivable for wedge-formed energy correction elements made of different materials to be used.

Due to the compact design of the energy correction unit 18, an excessive widening of the phase space of the beam of charged particles 6 can be avoided.

A collimator 20 is provided after the energy correction unit 18, viewed in the direction 12 of the beam of charged particles 6. This can for example be the collimator 20 shown in FIG. 1 or a part thereof, for example the collimator unit 20a.

In summary, the different aspects make it possible, by means of the small angle of deflection caused through the magnetic field of the scanning magnet 46, to achieve a large scanning area at the treatment location 7 and at the same time keep the distances of all the magnetic elements from the axis of rotation 14.

The efficiency of the particle beam therapy system shown in FIG. 1 can be determined by means of precise numerical Monte Carlo calculations. For example, the transmission efficiency at the treatment location 7 when the energy emitted by a cyclotron is reduced from 215 MeV to 90 MeV is still around 1%, and still 0.3% when reduced to 60 MeV. The width of the beam spot can thereby be adjusted to 4 mm (1σ) with very low ellipticity. The momentum width of the charged particles striking the treatment location 7 can be varied between 2 and 7 per thousand. The size of the beam spot is thereby maintained at the treatment location 7. Even with an energy of 250 MeV emitted by the beam generating unit and a reduction through the energy correction unit 18 to 90 MeV, the transmission efficiency is still up to 0.4%.

The physical bases of the interaction of protons with matter and the programs used to calculate the properties of beam guidance systems are described in the following scientific reports:
Particle Data Group, W.-M. Yao et al., “The Review of Particle Physics”, Journal of Physics G33 (2006) 1 and update 2008.
Karl L. Brown, Sam K. Howry, “TRANSPORT, A Computer Program for Designing Charged Particle Beam Transport Systems”, SLAC Report No. 91 (1970) and later updates of the TRANSPORT program by U. Rohrer and others.
U. Rohrer, “PSI Graphic TURTLE Framework based on a CERN-SLAC-FERMILAB version by K. L. Brown et al.”, http://aea.web.psi.ch/Urs_Rohrer/MyWeb/turtle.htm.
J. Drees, “Passage of Protons through Thick Degraders”, Cryoelectra Report September 2008.

The principle of an x-y scanning magnet is described in the following scientific report:
V. Anferov, “Combined X-Y scanning magnet for conformal proton radiation therapy”, Med. Phys. 32 (3), March 2005.

1. Beam guidance system for guiding a beam of charged particles
with a magnetic beam deflection unit,
wherein the magnetic beam deflection unit comprises an entry side for entry of the beam of charged particles into the magnetic beam deflection unit in a direction of entry,
wherein the magnetic beam deflection unit has an exit side for exit of the beam of charged particles from the magnetic beam deflection unit in a direction of exit, and
wherein the entry side of the magnetic beam deflection unit is, at least in sections, aligned substantially parallel to the exit side of the magnetic beam deflection unit.

2. Beam guidance system according to claim 1,
wherein the magnetic beam deflection unit is provided in the beam guidance system for deflection of the beam of charged particles such that the entry side lies at an oblique angle to the direction of entry of the beam of charged particles and/or the exit side lies at an oblique angle to the direction of exit of the beam of charged particles.

3. Beam guidance system according to claim 1,
wherein the magnetic beam deflection unit is provided in the beam guidance system for deflection of the beam of charged particles such that the direction of entry and the direction of exit are oriented at an angle of 30° to 60° relative to each other.

4. Beam guidance system according to claim 1,
wherein the magnetic beam deflection unit is a dipole magnet.

5. Beam guidance system according to claim 1,
wherein the beam guidance system includes several, in particular four magnetic beam deflection units.

6. Beam guidance system according to claim 1,
wherein the beam guidance system includes at least one magnetic beam forming unit, in particular several magnetic beam forming units, preferably in the form of one or more quadrupole magnets.

7. Beam guidance system according to claim 1,
wherein the beam guidance system includes a collimator unit before the first magnetic beam deflection unit, viewed in the direction of of the beam of charged particles.

8. Beam guidance system according to claim 1,
wherein the beam guidance system includes at least two magnetic beam deflection units and has a collimator unit between two of the at least two magnetic beam deflection units.

9. Beam guidance system according to claim 1,
wherein the beam guidance system at least in sections, designed to be movable, in particular rotatable.

10. Beam guidance system according to claim 1,
wherein the beam guidance system includes an energy correction unit for adjustment of the energy of the charged particles of the beam of charged particles.

11. Beam guidance system according to claim 10,
wherein the energy correction unit has at least one block-formed energy correction element which can be displaced transversely to the beam of charged particles and at least one wedge-formed energy correction element which can be displaced transversely to the beam of charged particles.

12. Beam guidance system according to claim 10,
wherein the energy correction unit has a plurality of block-formed energy correction elements displaceable transversely to the beam of charged particles which make possible different adjustments to the energy of the charged particles of the beam of charged particles.

13. Beam guidance system according to claim 10,
wherein the energy correction unit has several, in particular two wedge-formed energy correction elements displaceable transversely to the beam of charged particles.

14. Beam guidance system according to claim 10,
wherein the energy correction unit is at least partially manufactured of boron carbide.

15. Beam guidance system according to claim 10,
wherein the beam guidance system includes a collimator unit after the energy correction unit, viewed in the direction of the beam of charged particles.

16. Beam guidance system according to claim 10,
wherein the beam guidance system has a drift distance between the energy correction unit and the, viewed in the direction of the beam of charged particles, first magnetic beam deflection unit which is free of magnetic beam deflection units and/or magnetic beam forming units.

17. Particle beam therapy system, comprising:
a beam generating unit for generating a beam of charged particles, in particular ions, preferably protons, and
a beam guidance system according to claim 1,
wherein a beam forming unit is preferably provided after the beam generating unit, viewed in the direction of the beam of charged particles.

18. Method comprising the steps of:
generation of a beam of charged particles, in particular ions, preferably protons,
guidance of the beam of charged particles by means of a beam guidance system according to claim 1.

19. Beam guidance system according to claim 1,
wherein the magnetic beam deflection unit is provided in the beam guidance system for deflection of the beam of charged particles such that the direction of entry and the direction of exit are oriented at an angle of 40° to 50° relative to each other.

20. Beam guidance system according to claim 1,
wherein the magnetic beam deflection unit is provided in the beam guidance system for deflection of the beam of charged particles such that the direction of entry and the direction of exit are oriented at an angle of substantially 45° relative to each other.
